(19) United States
(12) Patent Application Publication
(10) Pub. No.: US 2025/0209709 A1
(43) Pub. Date: Jun. 26, 2025

(54) REACTION GENERATION APPARATUS, REACTION GENERATION METHOD, VIRTUAL PERSON PRESENTATION SYSTEM, AND NON-TRANSITORY COMPUTER READABLE MEDIUM

(71) Applicant: Yokogawa Electric Corporation, Tokyo (JP)

(72) Inventors: Kiyomi MURAOKA, Tokyo (JP); Motohiko ISHIDA, Tokyo (JP); Hirotaka YAMASHITA, Tokyo (JP); Takashi TSUBOTA, Tokyo (JP)

(21) Appl. No.: 18/916,699

(22) Filed: Oct. 16, 2024

(30) Foreign Application Priority Data
Dec. 26, 2023 (JP) 2023-219311

(51) Int. Cl. G06T 13/40; G06F 3/01

(52) U.S. Cl. CPC G06T 13/40; G06F 3/015; G06F 2203/011

(57) ABSTRACT

Provided is a reaction generation apparatus comprising an information acquisition unit that acquires subject input information input by a subject and subject brain wave information of the subject when the subject input information is input, a state generation unit that generates subject state information representing a state of the subject, based on the subject brain wave information, and a reaction generation unit that generates reaction information representing a reaction of the subject, based on the subject input information and the state of the subject. The information acquisition unit may further acquire biological information of the subject when the subject input information is input. The state generation unit may generate the subject state information based on the subject brain wave information and the biological information.

The contents of the following patent application(s) are incorporated herein by reference:

NO. 2023-219311 filed in JP on Dec. 26, 2023

### BACKGROUND

### 1. Technical Field

The present invention relates to a reaction generation apparatus, a reaction generation method, a virtual person presentation system, and a non-transitory computer readable medium.

### 2. Related Art

Non-Patent Document 1 describes “an attempt to bring a deceased person back to life through artificial intelligence (AI) or humanoid robot technique”.

### RELATED ART DOCUMENT

### Non-Patent Document

Non-Patent Document 1: Institute for Information and Communications Policy 2021 Vol. 5-1, p.131-144

### BRIEF DESCRIPTION OF THE DRAWINGS

FIG. 1 illustrates an example of a situation in which subject input information 114 is input to a subject 110.

FIG. 2 illustrates another example of a situation in which the subject input information 114 is input to the subject 110.

FIG. 3 is a block diagram illustrating an example of a reaction generation apparatus 100 according to an embodiment of the present invention.

FIG. 4 illustrates an example of a conversation between a user 120 and a virtual person model 130.

FIG. 5 illustrates an example of an electroencephalograph 14 that is capable of measuring subject brain wave information Ib1 or user brain wave information Ib2.

FIG. 6 illustrates an example of subject state information Is1.

FIG. 7 illustrates an example of user state information Is2.

FIG. 8 illustrates another example of a conversation between the user 120 and the virtual person model 130.

FIG. 9 illustrates another example of a conversation between the user 120 and the virtual person model 130.

FIG. 10 illustrates an example of a situation in which the subject 110 and the virtual person model 130 are communicating with a communication target 112.

FIG. 11 illustrates an example of a reaction inference model 62.

FIG. 12 illustrates another example of the reaction inference model 62.

FIG. 13 illustrates an example of a state inference model 66.

FIG. 14 illustrates an example of a relationship between the virtual person model 130 and a plurality of users 120.

FIG. 15 illustrates an example of an awkward state inference model 69.

FIG. 16 is a block diagram illustrating an example of a virtual person presentation system 200.

FIG. 17 is a flowchart illustrating an example of a reaction presentation method according to an embodiment of the present invention.

FIG. 18 illustrates an example of a computer 2200 in which the reaction generation apparatus 100 or the virtual person presentation system 200 according to an embodiment of the present invention may be embodied wholly or in part.

### DESCRIPTION OF EXEMPLARY EMBODIMENTS

Hereinafter, the present invention will be described through embodiments of the invention, but the following embodiments do not limit the invention according to the claims. In addition, not all of the combinations of features described in the embodiments are essential to the solution of the invention.

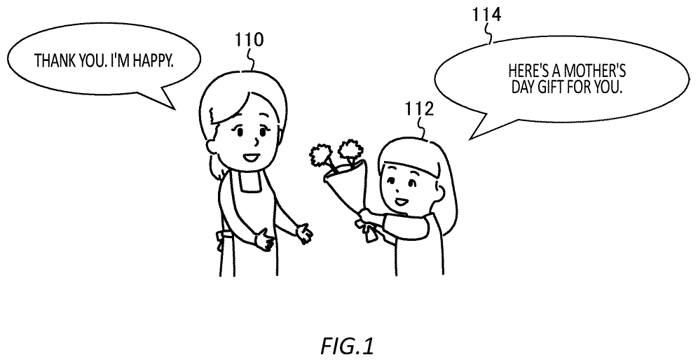

FIG. 1 illustrates an example of a situation in which subject input information 114 is input to a subject 110. The subject input information 114 is information that is input to subject 110 which may affect a state S1 (described below) of the subject 110. The subject input information 114 may be voice, may be movement of a communication target 112 that is visually recognized by the subject 110, or may be an image. Said image may be a motion image or may be a still image. The subject input information 114 may be a scenery, a landscape, a sight or a scene.

In the present example, the subject 110 is having a conversation with the communication target 112. In the present example, the communication target 112 is a human. The communication target 112 may be a robot with artificial intelligence. In the present example, the subject input information 114 is a speaking voice of the communication target 112 saying “Here is a Mother's day gift for you,” and a movement of the communication target 112 giving a flower bouquet. In the present example, the subject 110 is in a happy state S1 (described below) by the subject input information 114 saying “Here is a Mother's day gift for you.”

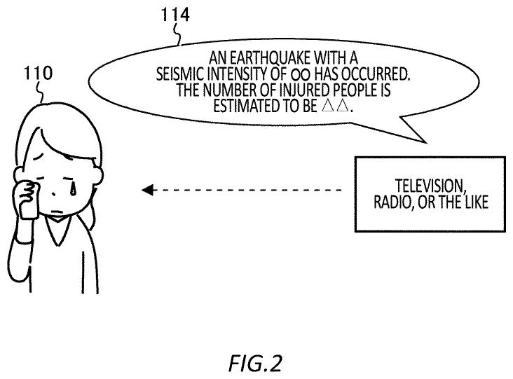

FIG. 2 illustrates another example of a situation in which the subject input information 114 is input to the subject 110. In the present example, the subject 110 is listening to a heartbreaking news of a disaster or the like being broadcasted by television, radio or the like. In the present example, the subject input information 114 is voice from the television, radio or the like. In the present example, the subject 110 is in a sad state S1 (described below) from listening to the voice from the television, the radio or the like broadcasting the disaster.

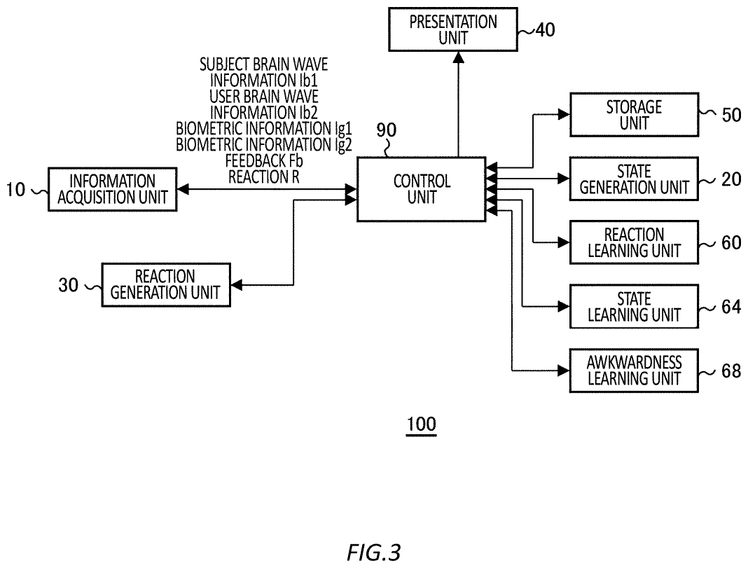

FIG. 3 is a block diagram illustrating an example of a reaction generation apparatus 100 according to an embodiment of the present invention. The reaction generation apparatus 100 comprises an information acquisition unit 10, a state generation unit 20, and a reaction generation unit 30. The reaction generation apparatus 100 may comprise a presentation unit 40, a storage unit 50, a reaction learning unit 60, a state learning unit 64, an awkwardness learning unit 68, and a control unit 90.

The reaction generation apparatus 100 may be realized in part or wholly by a computer. The control unit 90 may be a Central Processing Unit (CPU) of the computer. When the reaction generation apparatus 100 is realized by the computer, the computer may have installed thereon a reaction generation program to cause the computer to function as the reaction generation apparatus 100, or may have installed thereon an information processing program to cause the computer to perform an information processing method described below.

The information acquisition unit 10 acquires the subject input information 114 and subject brain wave information of the subject 110 when the subject input information 114 is input. The subject brain wave information is referred to as subject brain wave information Ib1. The information acquisition unit 10 may acquire the subject brain wave information Ib1 before and after the subject input information 114 is input. The subject brain wave information Ib1 after the subject input information 114 is input may refer to the subject brain wave information Ib1 when the subject input information 114 is input.

The information acquisition unit 10 may acquire user brain wave information of a user 120 (described below). The user brain wave information is referred to as user brain wave information Ib2.

The subject brain wave information Ib1 may be information reproducing at least a part of a temporal waveform of the brain wave of the subject 110. The subject brain wave information Ib1 may include data obtained by sampling the temporal waveform of the brain wave, may include data representing a magnitude of frequency component of the brain wave in one or more frequencies, or may include other data. The same may also apply to the user brain wave information Ib2. The subject brain wave information Ib1 may include data representing a magnitude of a component of at least one of a delta wave (less than 4 Hz), a theta wave (4 Hz or more and less than 8 Hz), an alpha wave (8 Hz or more and less than 14 Hz), a beta wave (14 Hz or more and less than 26 Hz), or gamma wave (26 Hz or more and less than 40 Hz). The same may also apply to the user brain wave information Ib2.

The alpha wave may be further classified according to frequency bands into a low alpha wave (8 Hz or more and less than 10 Hz), a medium alpha wave (10 Hz or more and less than 12 Hz), and a high alpha wave (12 Hz or more and less than 14 Hz). The subject brain wave information Ib1 and the user brain wave information Ib2 may include data that represents a magnitude of at least one of the low alpha wave, the medium alpha wave, or the high alpha wave. The beta wave may be further classified according to frequency bands into a low beta wave (14 Hz or more and less than 18 Hz) and a high beta wave (18 Hz or more and less than 26 Hz). The subject brain wave information Ib1 and the user brain wave information Ib2 may include data that represents a magnitude of at least one of the low beta wave or the high beta wave.

The subject brain wave information Ib1 may include temporal waveform information of one or more brain waves measured at one or more locations at a head portion including the head and face of the subject 110. The same may also apply to the user brain wave information Ib2. For example, the subject brain wave information Ib1 may be acquired by measuring a temporal waveform of a potential of an electrode arranged in an equally spaced manner in a vicinity of a scalp of the subject 110, such as in the International 10-20 system, or may be acquired through another method. The same may also apply to the user brain wave information Ib2. The intervals of a plurality of electrodes that are arranged on the scalp may not be equal. Said electrode may be provided on a wearable appliance to be worn on a head of the subject 110, such as a head gear, a head phone, an ear phone, eye glasses, or the like. The subject brain wave information Ib1 may be information acquired through wireless communication of electric signals in the electrode embedded in the body of the subject 110. The same may also apply to the user brain wave information Ib2.

A sum of amplitudes of the alpha wave, the beta wave, the theta wave, the gamma wave, and the delta wave at certain timing is referred to as a total amplitude As. As an example, when the proportion of the amplitude of the delta wave of the subject 110 in the total amplitude As is greater than any of the proportion of the amplitude of the alpha wave in the total amplitude As, the proportion of the amplitude of the beta wave in the total amplitude As, the proportion of the amplitude of the theta wave in the total amplitude As, and the proportion of the amplitude of the gamma wave in the total amplitude As, it can be presumed that the subject 110 is in a sleeping state. The same applies to the user 120.

A state of the subject 110 is referred to as a state S1. Information representing the state S1 of the subject 110 is referred to as subject state information Is1. The state generation unit 20 generates the subject state information Is1 based on the subject brain wave information Ib1. A state of the user 120 (described below) is referred to as a state S2. Information representing the state S2 of the user 120 is referred to as user state information Is2. The state generation unit 20 may generate the user state information Is2 based on the user brain wave information Ib2.

As an example, when the proportion, in the total amplitude As, of the amplitude of the theta wave of the subject 110 after the subject input information 114 is input is greater than the proportion, in the total amplitude As, of the amplitude of the theta wave before the subject input information 114 is input, the subject 110 may be in the state S1 with increasing fatigue and sleepiness. The same may also apply to the state S2 of the user 120 (described below).

As an example, when the proportion, in the total amplitude As, of the amplitude of the gamma wave of the subject 110 after the subject input information 114 is input is greater than the proportion, in the total amplitude As, of the amplitude of the gamma wave before the subject input information 114 is input, the subject 110 may be in the state S1 receiving a lot of stimulation. The same may also apply to the state S2 of the user 120 (described below).

As an example, when the proportion, in the total amplitude As, of a sum of the amplitude of the low alpha wave and the amplitude of the medium alpha wave of the subject 110 after the subject input information 114 is input is greater than the proportion, in the total amplitude As, of a sum of the amplitude of the low alpha wave and the amplitude of the medium alpha wave before the subject input information 114 is input, the subject 110 may be in the state S1 in which the relaxation degree is increased. The same may also apply to the state S2 of the user 120 (described below).

As an example, when the proportion, in the total amplitude As, of a sum of the amplitude of the high alpha wave and the amplitude of the low beta wave of the subject 110 after the subject input information 114 is input is greater than the proportion, in the total amplitude As, of a sum of the amplitude of the high alpha wave and the amplitude of the low beta wave before the subject input information 114 is input, the subject 110 may be in the state S1 with good balance between relaxation and concentration. The state in which the balance between the relaxation and the concentration is good, is a so-called a state of immersion. The same may also apply to the state S2 of the user 120 (described below).

The reaction generation unit 30 generates reaction information representing a reaction of the subject 110 based on the subject input information 114 and the state S1 of the subject 110. Said reaction of the subject 110 is referred to as a reaction R. Reaction information representing the reaction R is referred to as reaction information Ir.

The subject input information 114, the subject brain wave information Ib1 when said subject input information 114 is input, the state S1 of the subject 110, and the reaction information Ir may be associated with one another. A plurality of pieces of subject input information 114, each piece of subject brain wave information Ib1 when each of the plurality of pieces of subject input information 114 is input, the state S1 of each of the subjects 110, and the reaction information Ir of each of the subjects 110 may be associated with one another. The subject input information 114, the subject brain wave information Ib1, the state S1, and the reaction information Ir associated with one another may be stored in the storage unit 50.

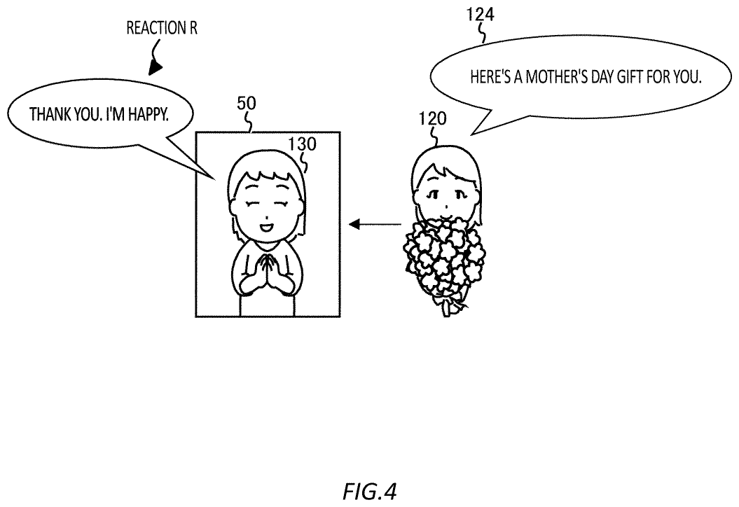

FIG. 4 illustrates an example of a conversation between the user 120 and the virtual person model 130. The virtual person model 130 is a virtual person model which simulates the subject 110. In the present example, the virtual person model 130 is presented on the presentation unit 40. The virtual person model 130 may be a robot. The user 120 is a person who has a conversation with the virtual person model 130. In the present example, user input information 124 of the user 120 is input to the reaction generation apparatus 100 (see FIG. 3).

Similarly to the subject input information 114, the user input information 124 may be voice, may be a movement of the user 120, or may be an image. The user input information 124 may be a scenery, a landscape, a sight or a scene. In the present example, the user input information 124 is a voice of the user 120 speaking “Here is a Mother's day gift for you,” and a movement of the user 120 giving a flower bouquet to the virtual person model 130.

The reaction generation unit 30 may generate the reaction information Ir corresponding to the user input information 124 based on the subject input information 114 and the reaction information Ir associated with each other and the user input information 124. The reaction R according to said reaction information Ir may be presented by the virtual person model 130. In the example of FIG. 1, the subject input information 114 saying “Here is a Mother's day gift for you” and the reaction information Ir of the subject 110 saying “Thank you. I am happy.” may be stored in the storage unit 50 in association with each other. In the present example, the virtual person model 130 presents a reaction R saying “Thank you. I am happy” from said subject input information 114 and said reaction information Ir stored in the storage unit 50 and the speech described above by the user 120.

The information acquisition unit 10 may acquire the user brain wave information Ib2 of the user 120 who came into contact with the reaction R presented by the virtual person model 130. In the present example, the information acquisition unit 10 acquires the user brain wave information Ib2 of the user 120 who came into contact with the reaction R of the virtual person model 130 saying “Thank you. I am happy.”

At the time the user 120 and the virtual person model 130 is having a conversation, the subject 110 may be deceased or may be alive. The user 120 and the communication target 112 (FIG. 1) may be the same person, or may be different persons.

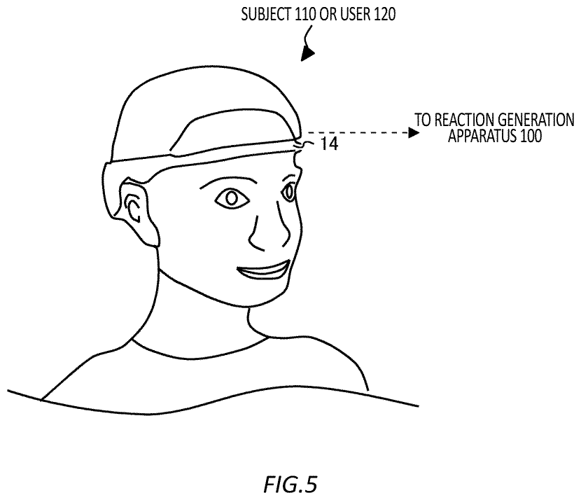

FIG. 5 illustrates an example of an electroencephalograph 14 that is capable of measuring subject brain wave information Ib1 or user brain wave information Ib2. The electroencephalograph 14 of the present example is of a headgear type. The electroencephalograph 14 may be of an earphone type. The subject brain wave information Ib1 or the user brain wave information Ib2 measured by the electroencephalograph 14 may be wirelessly transmitted to the reaction generation apparatus 100, or may be transmitted to the control unit 90 (see FIG. 3) of the reaction generation apparatus 100.

The subject 110 may have the subject input information 114 input thereto while wearing the electroencephalograph 14 of a headgear type or an earphone type. In this manner, the information acquisition unit 10 acquires the subject brain wave information Ib1 when the subject input information 114 is input. Similarly, the user 120 (described below) may have the user input information 124 (described below) input thereto while wearing the electroencephalograph 14 of a headgear type or an earphone type. In this manner, the information acquisition unit 10 acquires the user brain wave information Ib2 when the user input information 124 (described below) is input.

The information acquisition unit 10 may further acquire biological information of the subject 110 when the subject input information 114 is input. Said biological information is referred to as biological information Ig1. The biological information Ig1 may include at least one of heart rate information, perspiration amount information or body temperature information of the subject 110. The biological information Ig1 of the subject 110 may be acquired by a sensor provided on a wearable appliance worn by the subject 110. The state generation unit 20 may generate the subject state information Is1 based on the subject brain wave information Ib1 and the biological information Ig1.

The information acquisition unit 10 may further acquire biological information of the user 120 when the user input information 124 is input. Said biological information is referred to as biological information Ig2. The biological information Ig2 may include at least one of heart rate information, perspiration amount information or body temperature information of the user 120. The biological information Ig2 of the user 120 may be acquired by a sensor provided on a wearable appliance worn by the user 120. The state generation unit 20 may generate the user state information Is2 based on the user brain wave information Ib2 and the biological information Ig2.

A magnitude of a first power spectrum in the heartbeat of the subject 110 is referred to as LF1 and a magnitude of a second power spectrum is referred to as HF1. A magnitude of a first power spectrum in the heartbeat of the user 120 is referred to as LF2, and a magnitude of a second power spectrum is referred to as HF2. A frequency band of the second power spectrum is a band in which a frequency is higher than that in a frequency band of the first power spectrum. The frequency band of the first power spectrum and the frequency band of the second power spectrum may not overlap each other. The frequency band of the first power spectrum is, for example, 0.04 Hz to 0.15 Hz. The frequency band of the second power spectrum is, for example, 0.15 Hz to 0.4 Hz.

The state generation unit 20 may generate the subject state information Is 1 based on a change from the subject brain wave information Ib1 before the subject input information 114 is input to the subject brain wave information Ib1 after the subject input information 114 is input and the biological information Ig1. A change from a proportion, in the total amplitude As, of an amplitude of a brain wave in a predetermined frequency band in the subject brain wave information Ib1 before the subject input information 114 is input to a proportion, in the total amplitude As, of an amplitude of a brain wave in a predetermined frequency band in the subject brain wave information Ib1 after the subject input information 114 is input is referred to as a change C1. The state generation unit 20 may generate the subject state information Is1 based on the change C1 and a ratio of LF1 to HF1 (LF1/HF1).

The ratio of LF1 to HF1 (LF1/HF1) after the subject input information 114 is input is referred to as a ratio Rag1. A predetermined threshold of the ratio Rag1 is referred to as a threshold Pth1. The ratio of LF2 to HF2 (LF2/HF 2) after the user 120 has come into contact with the reaction R is referred to as ratio Rag2. A predetermined threshold of the ratio Rag2 is referred to as a threshold Pth2.

As an example, when the proportion, in the total amplitude As, of a sum of the amplitude of the high beta wave and the amplitude of the gamma wave of the subject 110 after the subject input information 114 is input is greater than the proportion, in the total amplitude As, of a sum of the amplitude of the high beta wave and the amplitude of the gamma wave before the subject input information 114 is input, and when the ratio of LF1 to HF1 (LF1/HF1) after the subject input information 114 is input is equal to or greater than the threshold Pth1, it can be presumed that an irritated state, a oversensitive state, or a stressed state of the subject 110 is increasing. When the ratio of LF1 to HF1 (LF1/HF1) is equal to or greater than the threshold Pth1, the subject 110 may be determined be in a state where the sympathetic nerve is predominant over the parasympathetic nerve. When the ratio of LF1 to HF1 (LF1/HF1) is less than the threshold Pth1, the subject 110 may be determined in a state where the parasympathetic nerve is predominant over the sympathetic nerve. The threshold Pth1 may be 2, 3, 4, or 5.

As an example, when the proportion, in the total amplitude As, of a sum of the amplitude of the high beta wave and the amplitude of the gamma wave of the subject 110 after the subject input information 114 is input is greater than the proportion, in the total amplitude As, of a sum of the amplitude of the high beta wave and the amplitude of the gamma wave before the subject input information 114 is input, and when the ratio of LF1 to HF1 (LF1/HF1) after the subject input information 114 is input is less than the threshold Pth1, it can be presumed that an excited state of the subject 110 is increasing.

A change from a proportion, in the total amplitude As, of an amplitude of a brain wave in a predetermined frequency band in the user brain wave information Ib2 before the user 120 comes into contact with the reaction R to a proportion, in the total amplitude As, of an amplitude of a brain wave in a predetermined frequency band in the user brain wave information Ib2 after the user 120 comes into contact with the reaction R is referred to as a change C2. The state generation unit 20 may generate the user state information Is2 based on the change C2 and a ratio of LF2 to HF2 (LF2/HF2). The state generation unit 20 may generate the subject state information Is2 based on the change C2 and the ratio of LF2 to HF2 (LF2/HF2).

The state generation unit 20 may generate the subject state information Is1 based on a magnitude relationship between the ratio Rag1 and the threshold Pth1 and the change C1. The state generation unit 20 may generate the user state information Is2 based on a magnitude relationship between the ratio Rag2 and the threshold Pth2 and the change C2.

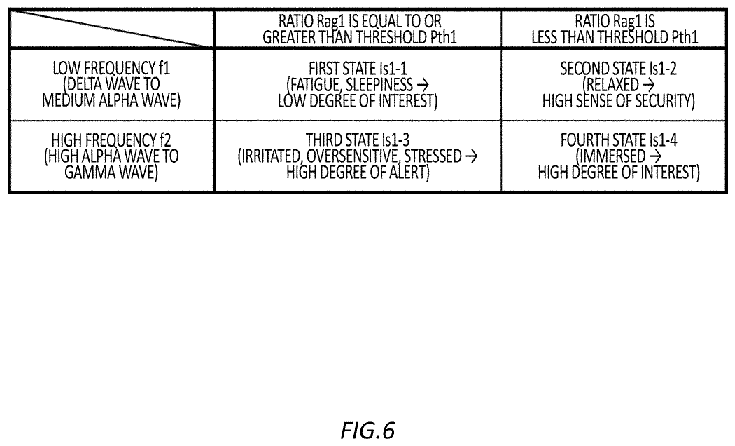

FIG. 6 illustrates an example of subject state information Is1. The subject state information Is 1 may include information according to a plurality of states (a first state Is1-1 to an n^{th} state Is1-n) of the subject 110. In the present example, the subject state information Is1includes information according to four states (the first state Is1-1 to a fourth state Is1-4) of the subject 110. In FIG. 6, the brainwave of a low frequency f1 refers to at least one of the delta wave, the theta wave, the low alpha wave, or the medium alpha wave; and the brainwave of a high frequency f2 refers to at least one of the high alpha wave, the low beta wave, high beta wave, or the gamma wave.

The amplitude of a brain wave of the subject 110 in a predetermined frequency band is referred to as amplitude Af1. The amplitude Af1 of the brain wave of the subject 110 before the subject input information 114 is input is referred to as amplitude Af1-1. The amplitude Af1 of the brain wave of the subject 110 after the subject input information 114 is input is referred to as an amplitude Af1-2. The brain wave in the predetermined frequency band may be at least one of the low alpha wave, the medium alpha wave, the high alpha wave, the low beta wave, the high beta wave, the gamma wave, or the theta wave.

The state generation unit 20 may generate the subject state information Is1 based on a change from a proportion of the amplitude Af1-1 in the total amplitude As to a proportion of the amplitude Af1-2 in the total amplitude As and a ratio of LF1 to HF1 (LF1/HF1). Said subject state information Is 1 may be state information Is1 according to one state (any of the first state Is1-1 to the n^{th} state Is1-n) of the plurality of states of the subject 110.

In the present example, the first state Is1-1 is a state of the subject 110 in a case where the proportion of the amplitude Af1-2 in the total amplitude As is greater than a proportion of the amplitude Af1-1 in the total amplitude As in the brain wave of a low frequency f1 and the ratio of LF1 to HF1 (LF1/HF1) after the subject input information 114 is input is equal to or greater than the threshold. When the subject 110 is the first state Is1-1, it can be presumed that a fatigue state or a sleepy state of the subject 110 is increasing. When the subject 110 is in the first state Is1-1, the state generation unit 20 may generate the state S1 in which the degree of interest of the subject 110 to at least one of the subject input information 114 or the communication target 112 is falling.

In the present example, the second state Is1-2 is a state of the subject 110 in a case where the proportion of the amplitude Af1-2 in the total amplitude As is greater than a proportion of the amplitude Af1-1 in the total amplitude As in the brain wave of a low frequency f1 and the ratio of LF1 to HF1 (LF1/HF1) after the subject input information 114 is input is less than the threshold. When the subject 110 is the second state Is1-2, it can be presumed that a relaxed state of the subject 110 is increasing. When the subject 110 is in the second state Is1-2, the state generation unit 20 may generate the state S1 in which a sense of security of the subject 110 toward at least one of the subject input information 114 or the communication target 112 is increasing.

In the present example, the third state Is1-3 is a state of the subject 110 in a case where the proportion of the amplitude Af1-2 in the total amplitude As is greater than a proportion of the amplitude Af1-1 in the total amplitude As in the brain wave of a high frequency f2 and the ratio of LF1 to HF1 (LF1/HF1) after the subject input information 114 is input is equal to or greater than the threshold. When the subject 110 is the third state Is1-3, it can be presumed that an irritated state, an oversensitive state, or a stressed state of the subject 110 is increasing. When the subject 110 is in the third state Is1-3, the state generation unit 20 may generate the state S1 in which the degree of alert of the subject 110 to at least one of the subject input information 114 or the communication target 112 is increasing.

In the present example, the fourth state Is1-4 is a state of the subject 110 in a case where the proportion of the amplitude Af1-2 in the total amplitude As is greater than a proportion of the amplitude Af1-1 in the total amplitude As in the brain wave of a high frequency f2 and the ratio of LF1 to HF1 (LF1/HF1) after the subject input information 114 is input is less than the threshold. When the subject 110 is the fourth state Is1-4, it can be presumed that an immersed state of the subject 110 is increasing. When the subject 110 is in the fourth state Is1-4, the state generation unit 20 may generate the state S1 in which the degree of interest of the subject 110 to at least one of the subject input information 114 or the communication target 112 is increasing.

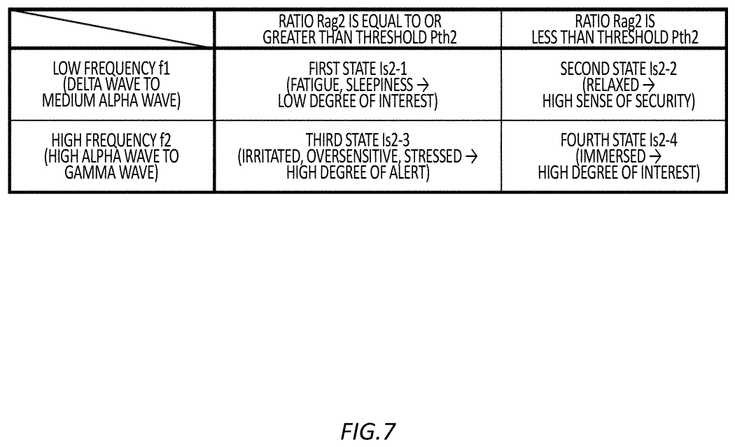

FIG. 7 illustrates an example of user state information Is2. Similarly to the subject state information Is1, the user state information Is2 may include information according to a plurality of states (a first state Is2-1 to an n^{th} state Is2-n) of the user 120. In the present example, the user state information Is2 includes information according to four states (the first state Is2-1 to a fourth state Is2-4) of the user 120.

The amplitude of a brain wave of the user 120 in a predetermined frequency band is referred to as amplitude Af2. The amplitude Af2 of the brain wave of the user 120 before the user 120 comes into contact with the reaction R (see FIG. 4) is referred to as amplitude Af2-1. The amplitude Af2 of the brain wave of the user 120 after the user 120 comes into contact with the reaction R is referred to as amplitude Af2-2.

Similarly to the subject state information Is1, the state generation unit 20 may generate the user state information Is2 based on a change from a proportion of the amplitude Af2-1 in the total amplitude As to a proportion of the amplitude Af2-2 in the total amplitude As and a ratio of LF2 to HF2 (LF2/HF2). Said user state information Is 2 may be state information Is2 according to one state (any of the first state Is2-1 to the n^{th} state Is2-n) of the plurality of states of the user 120.

Similar to the case of FIG. 6, when the user 120 is in the first state Is2-1, the state generation unit 20 may generate the state S2 in which the degree of interest of the user 120 to the reaction R is falling. When the user 120 is in the second state Is2-2, the state generation unit 20 may generate the state S2 in which a sense of security of the user 120 to the reaction R is increasing. When the user 120 is in the third state Is2-3, the state generation unit 20 may generate the state S2 in which the degree of alert of the user 120 to the reaction R is increasing. When the user 120 is in the fourth state Is2-4, the state generation unit 20 may generate the state S2 in which the degree of interest of the user 120 to the reaction R is increasing.

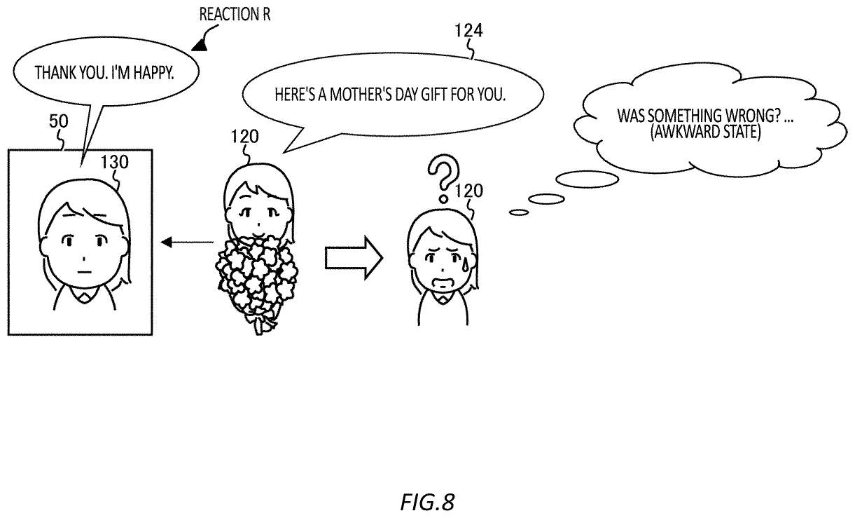

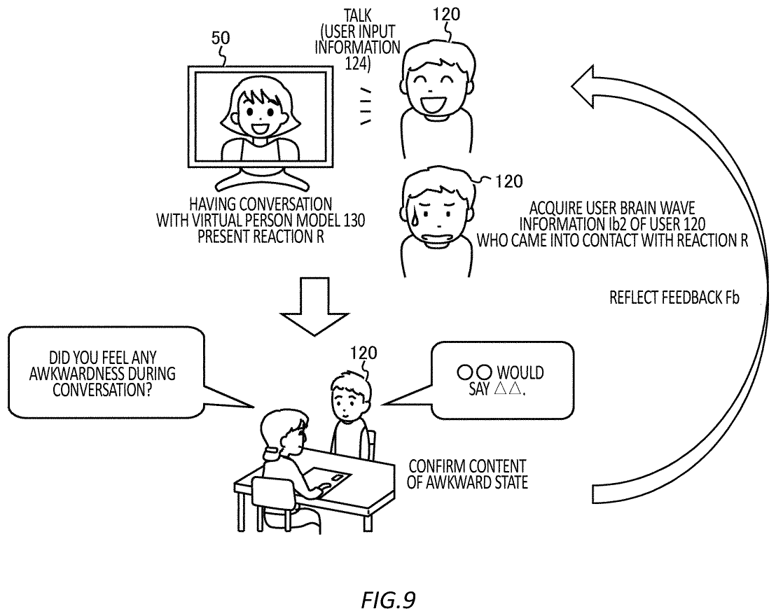

FIG. 8 and FIG. 9 illustrate other examples of a conversation between the user 120 and the virtual person model 130. FIG. 8 and FIG. 9 illustrate examples of the state S2 of the user 120 after the user 120 came into contact with the reaction R presented by the virtual person model 130. In the example of FIG. 8, similar to the example of FIG. 4, the user 120 is inputting the user input information 124 saying “Here is a Mother's day gift for you.” to the reaction generation apparatus 100 (see FIG. 3). In the present example, similar to the example of FIG. 4, the virtual person model 130 is presenting a speech saying “Thank you. I am happy.”

However, in the example of FIG. 8, the virtual person model 130 presents a reaction R with a blank expression compared to the example of FIG. 4. In the example of FIG. 8, the user 120 is in an awkward state thinking “Was something wrong?” towards this reaction R.

In the example of FIG. 9, the user 120 is inputting the user input information 124 to the reaction generation apparatus 100 (see FIG. 3). The virtual person model 130 is presenting the reaction R towards the user input information 124. In the example of FIG. 9, the user input information 124 is a talk to the virtual person model 130. In the example of FIG. 9, the user 120 who came into contact with said reaction R is in an awkward state.

In the examples of FIG. 8 and FIG. 9, the information acquisition unit 10 acquires the user brain wave information Ib2 of the user 120 who came into contact with the reaction R presented by the virtual person model 130. The state generation unit 20 generates the user state information Is2 representing the state S2 of the user 120 based on the user brain wave information Ib2. In the examples of FIG. 8 and FIG. 9, the state generation unit 20 generates the user state information Is2 that awkwardness against the reaction R is increasing, based on said user brain wave information Ib2. The state generation unit 20 may generate the user state information Is2 that the awkwardness is increasing when the user 120 is in the first state Is2-1 or the third state Is2-3 illustrated in FIG. 7.

When the user state information Is2 is a predetermined awkward state, the information acquisition unit 10 may acquire, from the user 120, a feedback for the reaction R presented by the virtual person model 130. Said feedback is referred to as a feedback Fb. As illustrated in FIG. 9, the user 120 who came into contact with the virtual person model 130 may be asked questions related to presence or absence of awkwardness to the reaction R or a content of awkwardness. The feedback Fb may be an answer of the user 120 to said questions. The user 120 may answer said questions in a spoken form or in a written form. The information acquisition unit 10 may acquire the answer in the spoken form or in the written form.

The reaction generation unit 30 may correct the reaction information Ir based on the feedback Fb. The reaction generation unit 30 may correct the reaction information Ir based on the feedback Fb such that the awkward state of the user 120 is decreased. The reaction generation unit 30 may correct the reaction information Ir based on the feedback Fb such that the state S2 of the user 120 becomes the second state Is2-2 or the fourth state Is2-4 illustrated in FIG. 7. In this manner, the reaction generation apparatus 100 can reduce the awkward state of the user 120.

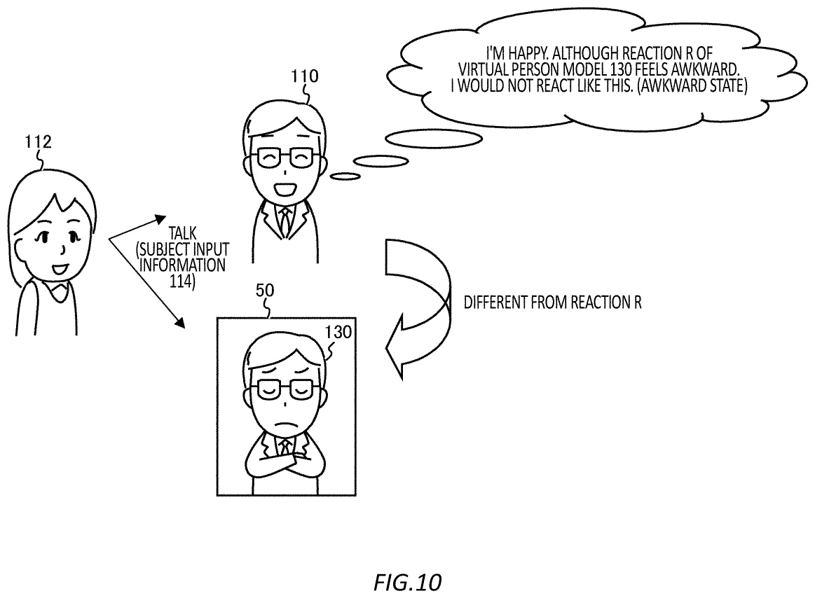

FIG. 10 illustrates an example of a situation in which the subject 110 and the virtual person model 130 are communicating with a communication target 112. The virtual person model 130 is a virtual person model which simulates the subject 110 themself. In the present example, the communication target 112 is talking to the subject 110 and the virtual person model 130 at the same timing. In the present example, the talking of the communication target 112 to the subject 110 and the virtual person model 130 is the subject input information 114. In the present example, while the subject 110 is feeling “happy” towards said talking, the virtual person model 130 is presenting the reaction R with a blank expression compared to the subject 110. In the present example, the subject 110 is in an awkward state against this reaction R.

In the present example, the information acquisition unit 10 acquires the subject brain wave information Ib1 of the subject 110 who came into contact with the reaction R presented by the virtual person model 130. In the present example, the state generation unit 20 generates the subject state information Is1 that the awkwardness against the reaction R is increasing, based on said subject brain wave information Ib1. The state generation unit 20 may generate the subject state information Is1 that the awkwardness is increasing when the subject 110 is in the first state Is1-1 or the third state Is1-3 illustrated in FIG. 6.

When the subject state information Is1 is a predetermined awkward state, the information acquisition unit 10 may acquire, from the subject 110, a feedback Fb for the reaction R presented by the virtual person model 130. The feedback Fb may be an answer of the subject 110 when being asked questions related to presence or absence of awkwardness against the reaction R. The subject 110 may answer said question in a written form or in a spoken form. The information acquisition unit 10 may acquire the answer in the written form or in the spoken form.

The reaction generation unit 30 may correct the subject state information Is1 based on the feedback Fb. The reaction generation unit 30 may correct the subject state information Is1 based on the feedback Fb such that the awkward state of the subject 110 is decreased. The reaction generation unit 30 may correct the subject state information Is1 based on the feedback Fb such that the state S1 of the subject 110 becomes the second state Is1-2 or the fourth state Is1-4 illustrated in FIG. 6. In this manner, the reaction generation apparatus 100 can reduce the awkward state of the subject 110.

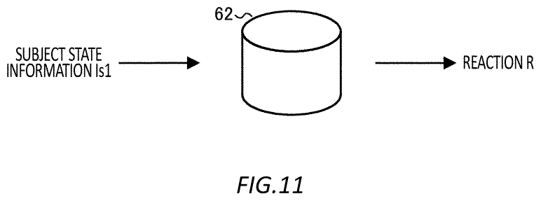

FIG. 11 illustrates an example of a reaction inference model 62. The information acquisition unit 10 may acquire the reaction R of the subject 110 presented by the virtual person model 130. The information acquisition unit 10 acquires the reaction R of the virtual person model 130 saying “Thank you. I am happy.” in the example of FIG. 4, for example. In the present example, the reaction learning unit 60 (see FIG. 3) learns the relationship between the subject state information Is1 and the reaction R of the subject 110 through machine learning. The reaction learning unit 60 generates the reaction inference model 62 through machine learning of the relationship between the subject state information Is1 and said reaction R. The reaction inference model 62 infers the reaction R of the subject 110 based on the subject state information Is1. Since the reaction inference model 62 has learned the relationship between the subject state information Is1 and the reaction R through machine learning, the reaction R may be inferred based on the subject state information Is1.

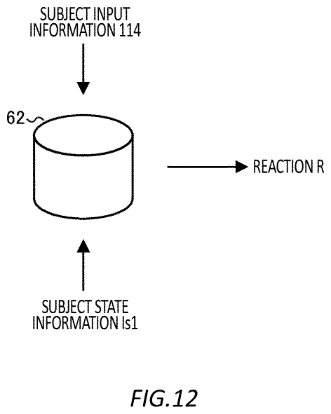

FIG. 12 illustrates another example of the reaction inference model 62. In the present example, the reaction learning unit 60 learns a relationship of the subject input information 114 (see FIG. 1) and the subject state information Is1 with the reaction R of the subject 110 through machine learning. The reaction learning unit 60 generates the reaction inference model 62 through machine learning of the relationship between the subject input information 114 and the subject state information Is1 and said reaction R. The reaction inference model 62 infers the reaction R of the subject 110 based on the subject input information 114 and the subject state information Is1. Since the reaction inference model 62 has learned the relationship of the subject state information Is1 and the subject input information 114 with the reaction R through machine learning, the reaction R may be inferred based on the subject input information 114 and the subject state information Is1.

The reaction learning unit 60 may correct the reaction inference model 62 based on the feedback Fb. The reaction learning unit 60 may correct the reaction inference model 62 based on the feedback Fb such that the awkward state of the subject 110 or the user 120 is decreased. The reaction learning unit 60 may correct the reaction inference model 62 based on the feedback Fb such that the state S1 of the subject 110 becomes the second state Is1-2 or the fourth state Is1-4 illustrated in FIG. 6. The reaction learning unit 60 may correct the reaction inference model 62 based on the feedback Fb such that the state S2 of the user 120 becomes the second state Is2-2 or the fourth state Is2-4 illustrated in FIG. 7. In this manner, the reaction generation apparatus 100 can reduce the awkward state of the subject 110 or the user 120.

In a case where the reaction inference model 62 infers the reaction R of the subject 110 based on one piece of the subject input information 114 and one piece of the subject state information Is1 and the information acquisition unit 10 acquires, from the subject 110 or the user 120, a feedback Fb that there is awkwardness in said reaction R, when the reaction learning unit 60 has already learned the relationship of the one piece of subject input information 114 and the one piece of subject state information Is1 with the reaction R, the reaction learning unit 60 may update the reaction inference model 62 based on the feedback Fb. In this manner, the reaction inference model 62 can infer the reaction R with higher accuracy based on the one piece of subject input information 114 and the one piece of subject state information Is1.

When the reaction learning unit 60 has not learned the relationship of the one piece of subject input information 114 and the one piece of subject state information Is1 with the reaction R, the reaction learning unit 60 may add, to the reaction inference model 62 as new teacher data, the one piece of subject input information 114 and the one piece of subject state information Is 1, and the reaction R based on the one piece of subject input information 114 and the one piece of subject state information Is1. The reaction learning unit 60 correcting the reaction inference model may refer to the reaction learning unit 60 updating the reaction inference model 62 and adding, to the reaction inference model 62, new teacher data.

In the examples of FIG. 11 or FIG. 12, the information acquisition unit 10 may acquire a first reaction of the subject 110 inferred by the reaction inference model 62 based on the one piece of subject input information 114 and a second reaction of said subject 110 when said one piece of subject input information 114 is input to said subject 110. The first reaction of the subject 110 is referred to as a first reaction R1, and the second reaction is referred to as a second reaction R2. The second reaction R2 is, for example, the reaction R of the subject 110 when the subject input information 114 (talking in the example of FIG. 10) is input to the subject 110 in the example of FIG. 10.

The reaction inference model 62 may correct the reaction inference model 62 based on the first reaction R1 and the second reaction R2. The reaction inference model 62 may correct the reaction inference model 62 based on the first reaction R1 and the second reaction R2. The first reaction R1 inferred by the reaction inference model 62 and the second reaction R2 of the subject 110 when the subject input information 114 is input to the subject 110 may be different. The reaction inference model 62 may correct the reaction inference model 62 such that the first reaction R1 becomes closer to the second reaction R2. In this manner, the reaction inference model 62 can infer the first reaction R1 with higher accuracy.

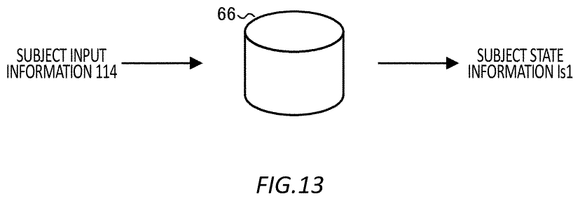

FIG. 13 illustrates an example of a state inference model 66. The state learning unit 64 (see FIG. 3) learns the relationship between the subject input information 114 and the subject state information Is1 through machine learning. The state learning unit 64 generates the state inference model 66 through machine learning of the relationship between the subject input information 114 and the subject state information Is1. The state inference model 66 infers the state S1 of the subject 110 based on the subject input information 114. Since the state inference model 66 has learned the relationship between the subject input information 114 and the subject state information Is1 through machine learning, the state S1 may be inferred based on the subject input information 114.

The state learning unit 64 may correct the state inference model 66 based on the feedback Fb. The state learning unit 64 may correct the state inference model 66 based on the feedback Fb such that the awkward state of the subject 110 or the user 120 is decreased. The state learning unit 64 may correct the state inference model 66 based on the feedback Fb such that the state S1 of the subject 110 becomes the second state Is1-2 or the fourth state Is1-4 illustrated in FIG. 6. The state learning unit 64 may correct the state inference model 66 based on the feedback Fb such that the state S2 of the user 120 becomes the second state Is2-2 or the fourth state Is2-4 illustrated in FIG. 7. In this manner, the reaction generation apparatus 100 can reduce the awkward state of the subject 110 or the user 120.

In a case where the state inference model 66 infers a one piece of subject state information Is1 based on a one piece of subject input information 114, the state learning unit 64 may update the state inference model 66 with the one piece of subject input information 114 and the one piece of subject state information Is 1 as new learning data. In this manner, the state inference model 66 can infer a one piece of subject state information Is1 with higher accuracy based on the one piece of subject input information 114.

When the state learning unit 64 has not learned the relationship between the one piece of subject input information 114 and the one piece of subject state information Is 1, the state learning unit 64 may add, to the state inference model 66, the one piece of subject input information 114 and the one piece of subject state information Is 1 as new teacher data.

The reaction learning unit 60 (see FIG. 3) may correct the reaction inference model 62 based on the state S of the subject 110 inferred by the state inference model 66. The reaction learning unit 60 may correct the reaction inference model 62 based on the inferred state S such that the state S1 of the subject 110 becomes the second state Is1-2 or the fourth state Is1-4 illustrated in FIG. 6. In this manner, the reaction generation apparatus 100 can reduce the awkward state of the subject 110 or the user 120.

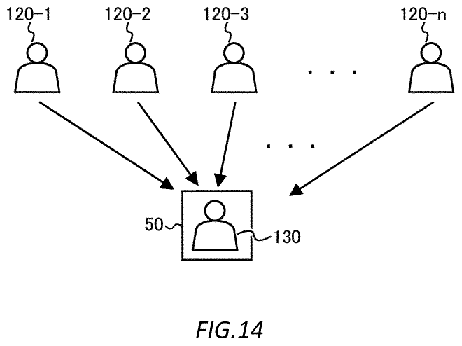

FIG. 14 illustrates an example of a relationship between the virtual person model 130 and a plurality of users 120. In the present example, each of the plurality of users 120 inputs the user input information 124 (see FIG. 8) to the reaction generation apparatus 100. The reaction generation unit 30 generates respective reactions R corresponding to each piece of user input information 124.

The reaction learning unit 60 (see FIG. 3) may generate a reaction inference model 62 for each user 120. The information acquisition unit 10 may have a face authentication unit that authenticates a face of the user 120. Said face authentication unit may identify one user 120 by face authentication. The reaction learning unit 60 may generate a reaction inference model 62 corresponding to said one user 120.

The information acquisition unit 10 may acquire an attribute of the user 120. An attribute of the user 120 refers to, for example, a degree of psychological safety of one user 120 (for example, the user 120-1) towards another user 120 (for example, the user 120-2). The degree of psychological safety may be divided into a plurality of steps. The degree of psychological safety is divided into two steps of “high” and “low”, for example. A case where psychological safety is high is where one user 120 can communicate with the virtual person model 130 without minding other users 120. In this case, the probability that the speech content of the one user 120 reflects real thoughts of the one user 120 is high. Other users 120 with high psychological safety are, for example, family, friends, or the like of the one user 120. A case where psychological safety is low is where the one user 120 communicates with the virtual person model 130 while minding other users 120. In this case, the probability that the speech content of the one user 120 does not reflect real thoughts of the one user 120 is high. Other users 120 with low psychological safety are, for example, strangers who are not acquainted with the one user 120.

The attributes of other users (for example, the user 120-2 to the user 120-n) when seen from the one user 120 (for example, the user 120-1) may be previously acquired and may be stored in the storage unit 50. The reaction learning unit 60 may generate the reaction inference model 62 based on the attribute of the user 120. In a case where the reaction learning unit 60 generates the reaction inference model 62 for each of two or more users 120, when another user 120 with low psychological safety as seen from one of the two or more users 120 is included in the two or more users 120, the reaction inference model 62 corresponding to said another user 120 may be generated. In this manner, the reaction inference model 62 can infer a reaction R that is acceptable for the one user 120 in a case where said another user 120 is present.

In a case where the reaction learning unit 60 generates the reaction inference model 62 for each user 120, the state learning unit 64 (see FIG. 3) may generate a state inference model 66 that is common for the plurality of users 120. The state inference model 66 infers the common state S1 irrespective of the user 120, and the reaction learning unit 60 learns the relationship of the subject state information Is1 according to said state S1 with the reaction R through machine learning, thereby facilitating the reaction learning unit 60 to generate the reaction inference model 62 for each user 120 with higher accuracy.

The reaction learning unit 60 may correct the reaction inference model 62 for each user 120. In a case where the reaction learning unit 60 corrects the reaction inference model 62 for each user 120, the state learning unit 64 may not correct the common state inference model 66. The common state inference model 66 has learned, through machine learning, the relationship between the plurality of pieces of subject input information 114 (see FIG. 1) and the subject state information Is1 corresponding to each of the plurality of pieces of subject input information 114. Thus, the common state inference model 66 can infer the state S1 which the subject 110 who came into contact with the subject input information 114 is most likely to be in. Thus, the reaction learning unit 60 corrects the reaction inference model 62 for each user 120 and the state learning unit 64 does not correct the common state inference model 66, thereby facilitating the reaction learning unit 60 to correct the reaction inference model 62 for each user 120 with higher accuracy.

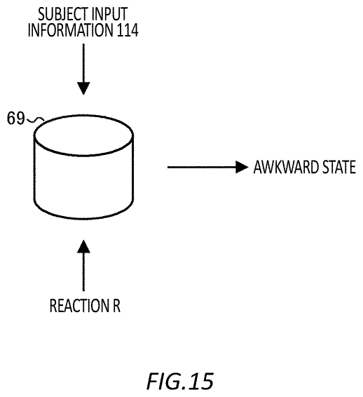

FIG. 15 illustrates an example of an awkward state inference model 69. In the present example, the awkwardness learning unit 68 (see FIG. 3) learns the relationship of the subject input information 114 (see FIG. 1) and the reaction R of the subject 110) with the awkward state of the user 120 through machine learning. In a case where the awkwardness learning unit 68 learns the relationship of the subject input information 114 and the reaction R of the subject 110 with the awkward state of the user 120 through machine learning, said reaction R of the subject 110 may be the reaction R inferred by the reaction inference model 62 in the example of FIG. 11 or FIG. 12.

The awkwardness learning unit 68 generates the awkward state inference model 69 through machine learning of the relationship of the subject input information 114 and the reaction R with said awkward state. The awkward state inference model 69 infers the awkward state of the user 120 based on the subject input information 114 and the reaction R. Since the awkward state inference model 69 has learned the relationship of the subject input information 114 and the reaction R with the awkward state through machine learning, the awkward state of the user 120 may be inferred based on the subject input information 114 and the reaction R.

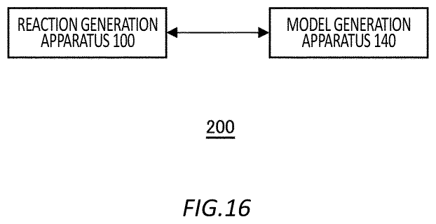

FIG. 16 is a block diagram illustrating an example of a virtual person presentation system 200. The virtual person presentation system 200 comprises a reaction generation apparatus 100 and a model generation apparatus 140. The model generation apparatus 140 generates the virtual person model 130 (see FIG. 4, FIG. 8 to FIG. 10, and FIG. 14).

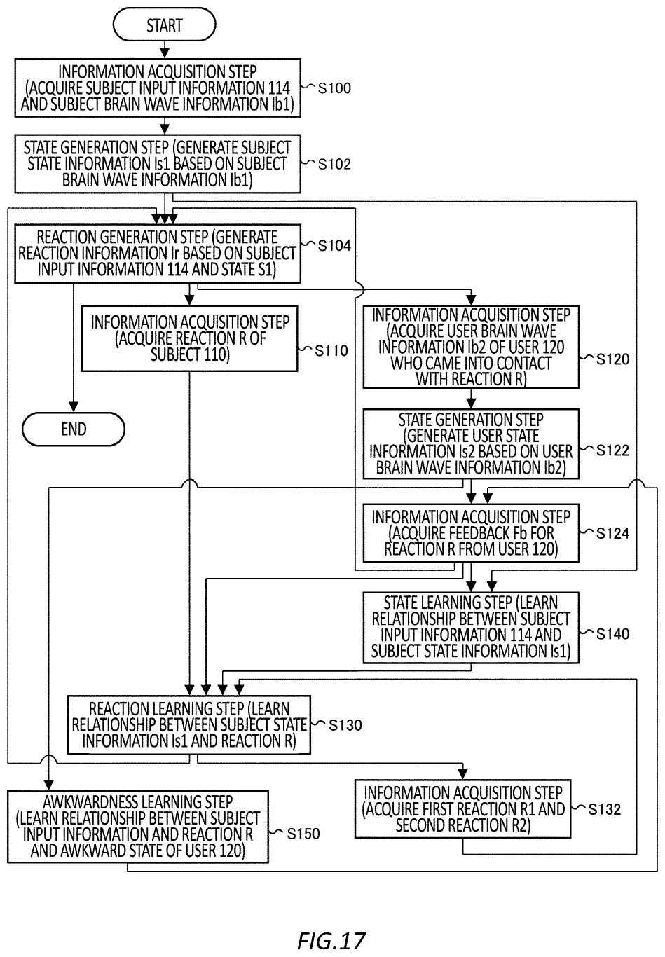

FIG. 17 is a flowchart illustrating an example of a reaction presentation method according to an embodiment of the present invention. The reaction presentation method comprises an information acquisition step S100, a state generation step S102, and a reaction generation step S104. The reaction presentation method may comprise an information acquisition step S110, an information acquisition step S120, a state generation step S122, an information acquisition step S124, a reaction learning step S130, an information acquisition step S132, a state learning step S140, and an awkwardness learning step S150. The reaction presentation method according to an embodiment of the present invention will be described using the example of the reaction generation apparatus 100 illustrated in FIG. 3.

The information acquisition step S100 is a step of acquiring, by the information acquisition unit 10, subject input information 114 input by the subject 110 and subject brain wave information Ib1 of the subject 110 when the subject input information 114 is input. The state generation step S102 is a step of generating, by the state generation unit 20, the subject state information Is1 representing a state S1 of the subject 110 based on the subject brain wave information Ib1. The reaction generation step S104 is a step of generating, by the reaction generation unit 30, reaction information Ir representing the reaction R of the subject 110 based on the subject input information 114 and the state S1 of the subject 110.

The information acquisition step S100 may be step of further acquiring, by the information acquisition unit 10, biological information Ig1 of the subject 110 when the subject input information 114 is input. The state generation step S102 may be a step of generating, by the state generation unit 20, subject state information Is1 based on the subject brain wave information Ib1 and the biological information Ig1.

The information acquisition step S100 may be a step of acquiring, by the information acquisition unit 10, subject brain wave information Ib1 before and after the subject input information 114 is input. The state generation step S102 may be a step of generating, by the state generation unit 20, subject state information Is1 based on a change from the subject brain wave information Ib1 before the subject input information 114 is input to the subject brain wave information Ib1 after the subject input information 114 is input and the biological information Ig1.

The state generation step S102 may be a step of generating, by the state generation unit 20, the subject state information Is1 based on the change C1 and a ratio of LF1 to HF1 (LF1/HF1). The state generation step S102 may be a step of generating, by the state generation unit 20, the subject state information Is1 based on a magnitude relationship between the ratio Rag1 and the threshold Pth1, and the change C1. The state generation step S102 may be a step of generating, by the state generation unit 20, the subject state information Is1 based on a change from a proportion of the amplitude Af1-1 in the total amplitude As to a proportion of the amplitude Af1-2 in the total amplitude As and a ratio of LF1 to HF1 (LF1/HF1).

The information acquisition step S120 is a step of acquiring, by the information acquisition unit 10, the user brain wave information Ib2 of the user 120 who came into contact with the reaction R presented by the virtual person model 130. The state generation step S122 is a step of generating, by the state generation unit 20, the user state information Is2 representing the state S2 of the user 120 based on the user brain wave information Ib2. The information acquisition step S124 is a step of acquiring, from the user 120 by the information acquisition unit 10, a feedback Fb to the reaction R presented by the virtual person model 130, when the user state information Is2 is a predetermined awkward state. The reaction generation step S104 is a step of correcting, by the reaction generation unit 30, at least one of the subject state information Is1 or the reaction information Ir based on the feedback Fb.

The information acquisition step S110 is a step of acquiring, by the information acquisition unit 10, the reaction R of the subject 110 presented by the virtual person model 130. The reaction learning step S 130 is a step of generating, by the reaction learning unit 60, the reaction inference model 62 for inferring the reaction R of the subject 110 based on the subject state information Is1 through machine learning of the relationship between the subject state information Is1 and the reaction R. The reaction learning step S130 may be a step of generating, by the reaction learning unit 60, the reaction inference model 62 for inferring the reaction R of the subject 110 based on the subject input information 114 and the subject state information Is1 through machine learning of the relationship of the subject input information 114 and the subject state information Is 1 with the reaction R. The reaction learning step S130 may be a step of correcting the reaction inference model 62 by the reaction learning unit 60 based on the feedback Fb acquired at the information acquisition step S124.

The state learning step S140 is a step of generating, by the state learning unit 64, the state inference model 66 for inferring the state S1 of the subject 110 based on the subject input information 114 through machine learning of the relationship between the subject input information 114 and the subject state information Is1. The state learning step S140 may be a step of correcting the state inference model 66 by the state learning unit 64 based on the feedback Fb acquired at the information acquisition step S124.

The reaction learning step S130 may be a step of correcting, by the reaction learning unit 60, the reaction inference model 62 based on the state S1 of the subject 110 inferred at the state learning step S140. The reaction learning step S130 may be a step of generating, by the reaction learning unit 60, the reaction inference model 62 for each user 120. The state learning step S140 may be a step of generating, by the state learning unit 64, the state inference model 66 that is common for the plurality of users 120.

The reaction learning step S130 may be a step of correcting, by the reaction learning unit 60, the reaction inference model 62 for each user 120. The state learning step S140 may be a step of not correcting, by the state learning unit 64, the state inference model 66 that is common for the plurality of users 120.

The information acquisition step S132 is a step of acquiring, by the information acquisition unit 10, the first reaction R1 of the subject 110 inferred by the reaction inference model 62 based on one piece of subject input information 114 and the second reaction R2 of the subject 110 when the one piece of subject input information 114 is input to said subject 110. The reaction learning step S130 may be a step of correcting, by the reaction learning unit 60, the reaction inference model 62 based on the first reaction R1 and the second reaction R2. The reaction generation step S104 may be a step of generating, by the reaction generation unit 30, the reaction information Ir according to the reaction R inferred by the reaction inference model 62.

The awkwardness learning step S150 is a step of generating, by the awkwardness learning unit 68, the awkward state inference model 69 for inferring the awkward state of the user 120 based on the subject input information 114 and the reaction R through machine learning of the relationship of the subject input information 114 and the reaction R of the subject 110 with the awkward state of the user 120. The information acquisition step S124 may be a step of acquiring, by the information acquisition unit 10 from the user 120, a feedback Fb to the reaction R presented by the virtual person model 130 when an awkward state is inferred by the awkward state inference model 69.

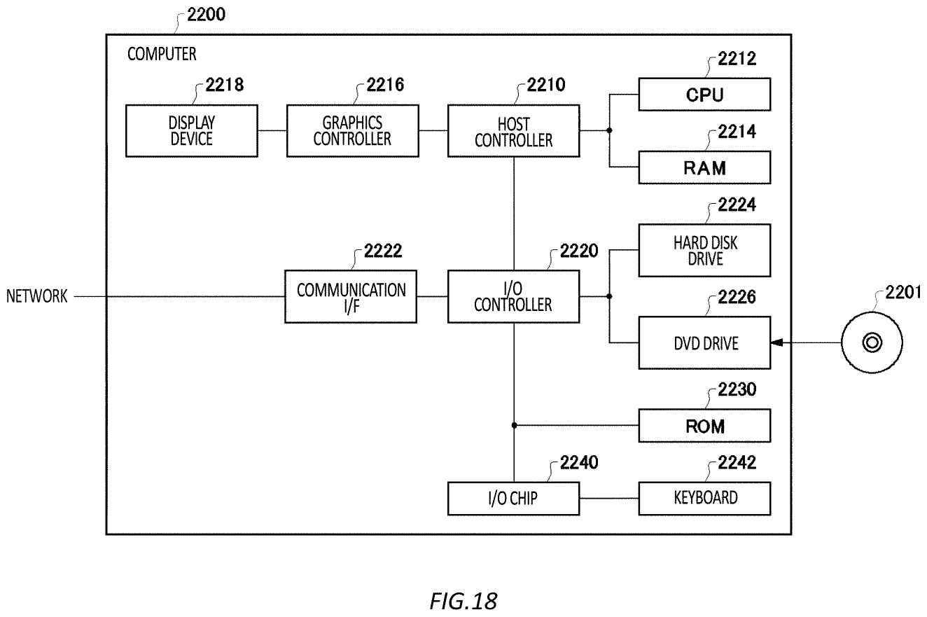

FIG. 18 illustrates an example of a computer 2200 in which the reaction generation apparatus 100 or the virtual person presentation system 200 according to an embodiment of the present invention may be embodied wholly or in part. The program installed on the computer 2200 can cause the computer 2200 to function as an operation associated with the reaction generation apparatus 100 or the virtual person presentation system 200 according to embodiments of the present invention or one or more sections of the reaction generation apparatus 100 or the virtual person presentation system 200, or can cause said operation or said one or more sections to be executed, or can cause the computer 2200 to execute each step (see FIG. 17) according to the method of the present invention. Such a program may be executed by a CPU 2212 to cause a computer 2200 to execute certain operations associated with some or all of the blocks of flowcharts (refer to FIG. 17) and block diagrams (refer to FIG. 3) described herein.

The computer 2200 according to an embodiment of the present invention includes the CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input/output unit such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input/output controller 2220. The computer further includes legacy input/output units such as a ROM 2230 and a keyboard 2242. The ROM 2230, the keyboard 2242, and the like are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 on a frame buffer or the like provided in the RAM 2214 or in the RAM 2214 itself to cause the image data to be displayed on the display device 2218.

The communication interface 2222 communicates with other electronic devices via a network. The hard disk drive 2224 stores programs and data used by the CPU 2212 in the computer 2200. The DVD-ROM drive 2226 reads the programs or the data from the DVD-ROM 2201, and provides the read programs or data to the hard disk drive 2224 via the RAM 2214. The IC card drive reads programs and data from an IC card, or writes programs and data to the IC card.

The ROM 2230 stores a boot program or the like executed by the computer 2200 at the time of activation, or a program depending on the hardware of the computer 2200. The input/output chip 2240 may connect various input/output unit via a parallel port, a serial port, a keyboard port, a mouse port, or the like to the input/output controller 2220.

Programs are provided by a computer readable medium such as the DVD-ROM 2201 or the IC card. The programs are read from the computer readable medium, are installed in the hard disk drive 2224, the RAM 2214, or the ROM 2230 which is also an example of the computer readable medium, and are executed by the CPU 2212. The information processing described in these programs is read by the computer 2200, and provides cooperation between the programs and the various types of hardware resources. An apparatus or method may be constituted by realizing the operation or processing of information in accordance with the use of the computer 2200.

For example, when a communication is executed between the computer 2200 and an external device, the CPU 2212 may execute a communication program loaded onto the RAM 2214 to instruct communication processing to the communication interface 2222, based on the processing described in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may execute various types of processing on the data on the RAM 2214. The CPU 2212 may then write back the processed data to the external recording medium.

Various types of information, such as various types of programs, data, tables, and databases, may be stored in the recording medium to undergo information processing. The CPU 2212 may execute various types of processing on the data read from the RAM 2214, which includes various types of operations, information processing, condition judging, conditional branch, unconditional branch, retrieval or replacement of information, or the like, as described throughout the present disclosure and designated by an instruction sequence of programs. The CPU 2212 may write the result back to the RAM 2214.

The CPU 2212 may retrieve information in a file, a database, or the like in the recording medium. For example, when a plurality of entries, each having an attribute value of a first attribute associated with an attribute value of a second attribute, are stored in the recording medium, the CPU 2212 may retrieve an entry matching the condition whose attribute value of the first attribute is designated, from among the plurality of entries, read the attribute value of the second attribute stored in the entry, and read a second attribute value to acquire the attribute value of the second attribute associated with the first attribute satisfying the predetermined condition.

The program or software modules described above may be stored in the computer readable media on the computer 2200 or of the computer 2200. A recording medium such as a hard disk or a RAM provided in a server system connected to a dedicated communication network or the Internet can be used as the computer readable media. The program may be provided to the computer 2200 by the recording medium.

While the present invention has been described by way of the embodiments, the technical scope of the present invention is not limited to the scope described in the above-described embodiments. It is apparent to persons skilled in the art that various alterations or improvements can be made to the above-described embodiments. It is also apparent from the described scope of the claims that the embodiments added with such alterations or improvements can be included the technical scope of the present invention.

The operations, procedures, steps, stages, or the like of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” for convenience in the claims, embodiments, or diagrams, it does not necessarily mean that the process must be performed in this order.

### EXPLANATION OF REFERENCES

10: information acquisition unit, 14: electroencephalograph, 20: state generation unit, 30: reaction generation unit, 40: presentation unit, 50: storage unit, 60: reaction learning unit, 62: reaction inference model, 64: state learning unit, 66: state inference model, 68: awkwardness learning unit, 69: awkward state inference model, 90: control unit, 100: reaction generation apparatus, 110: subject, 112: communication target, 114: subject input information, 120: user, 124: user input information, 130: virtual person model, 140: model generation apparatus, 200: virtual person presentation system, 2200: computer, 2201: DVD-ROM, 2210: host controller, 2212: CPU, 2214: RAM, 2216: graphics controller, 2218: display device, 2220: input/output controller, 2222: communication interface, 2224: hard disk drive, 2226: DVD-ROM drive, 2230: ROM, 2240: input/output chip, 2242: keyboard.

What is claimed is:

1. A reaction generation apparatus comprising:
an information acquisition unit that acquires subject input information input to a subject and subject brain wave information of the subject when the subject input information is input;
a state generation unit that generates subject state information representing a state of the subject, based on the subject brain wave information; and
a reaction generation unit that generates reaction information representing a reaction of the subject, based on the subject input information and the state of the subject.

2. The reaction generation apparatus according to claim 1, wherein
the information acquisition unit further acquires biological information of the subject when the subject input information is input, and
the state generation unit generates the subject state information based on the subject brain wave information and the biological information.

3. The reaction generation apparatus according to claim 2, wherein
the information acquisition unit acquires the subject brain wave information before and after the subject input information is input, and
the state generation unit generates the subject state information based on a change from the subject brain wave information before the subject input information is input to the subject brain wave information after the subject input information is input, and the biological information.

4. The reaction generation apparatus according to claim 3, wherein
the state generation unit generates the subject state information based on a change from a proportion, in a total amplitude, of an amplitude of brain wave at a predetermined frequency band in the subject brain wave information before the subject input information is input to a proportion, in the total amplitude, of an amplitude of brain wave at the frequency band in the subject brain wave information after the subject input information is input, and a ratio of a magnitude of a first power spectrum to a magnitude of a second power spectrum in a heartbeat of the subject,
the total amplitude is a sum of amplitudes of an alpha wave, a beta wave, a theta wave, a gamma wave, and a delta wave, and
a frequency band of the second power spectrum is a band that is of a higher frequency than a frequency band of the first power spectrum.

5. The reaction generation apparatus according to claim 4, wherein the state generation unit generates the subject state information based on a magnitude relationship of a ratio of a magnitude of the first power spectrum to a magnitude of the second power spectrum and a predetermined threshold of a ratio of the magnitude of the first power spectrum to the magnitude of the second power spectrum, and the change from the proportion, in the total amplitude, of an amplitude of the brain wave of the frequency band before the subject input information is input to the proportion, in the total amplitude, of an amplitude of the brain wave of the frequency band after the subject input information is input.

6. The reaction generation apparatus according to claim 4, wherein
the subject state information includes information according to a plurality of states of the subject, and
the state generation unit generates the subject state information according to one state among the plurality of states based on a ratio of a magnitude of the first power spectrum to a magnitude of the second power spectrum and the change from the proportion, in the total amplitude, of an amplitude of the brain wave the frequency band before the subject input information is input to the proportion, in the total amplitude, of an amplitude of the brain wave of the frequency band after the subject input information is input.

7. The reaction generation apparatus according to claim 6, wherein the brain wave of the frequency band is at least one of a delta wave, a theta wave, a low alpha wave, or a medium alpha wave, or at least one of a high alpha wave, a low beta wave, a high beta wave, or a gamma wave.

8. The reaction generation apparatus according to claim 2, wherein
the reaction of the subject is presented by a presentation unit that presents a virtual person model,
the information acquisition unit further acquires user brain wave information of a user who came into contact with the reaction presented by the virtual person model,
the state generation unit generates user state information indicating a state of the user based on the user brain wave information,
the information acquisition unit acquires, from the user, a feedback for the reaction presented by the virtual person model, when the user state information is a predetermined awkward state, and
the reaction generation unit corrects at least one of the subject state information or the reaction information based on the feedback.

9. The reaction generation apparatus according to claim 2, wherein
the reaction of the subject is presented by a presentation unit that presents a virtual person model, and
the information acquisition unit further acquires the reaction of the subject presented by the virtual person model,
the reaction generation apparatus further comprising a reaction learning unit that generates a reaction inference model for inferring a reaction of the subject based on the subject state information through machine learning of a relationship between the subject state information and the reaction of the subject acquired by the information acquisition unit.

10. The reaction generation apparatus according to claim 9, wherein
the information acquisition unit further acquires user brain wave information of a user who came into contact with the reaction presented by the virtual person model,
the state generation unit generates user state information representing a state of the user based on the user brain wave information,
the information acquisition unit acquires, from the user, a feedback for the reaction presented by the virtual person model, when the user state information is a predetermined awkward state, and
the reaction learning unit corrects the reaction inference model based on the feedback.

11. The reaction generation apparatus according to claim 9, further comprising:
a state learning unit that generates a state inference model for inferring a state of the subject based on the subject input information through machine learning of a relationship between the subject input information and the subject state information, wherein
the reaction learning unit corrects the reaction inference model based on the state of the subject inferred by the state inference model.

12. The reaction generation apparatus according to claim 10, further comprising:
a state learning unit that generates a state inference model for inferring a state of the subject based on the subject input information through machine learning of a relationship between the subject input information and the subject state information, wherein
the reaction learning unit generates the reaction inference model for each of a plurality of users, each being identical to the user, and
the state learning unit generates the state inference model that is common for the plurality of users.

13. The reaction generation apparatus according to claim 12, wherein
the reaction learning unit corrects the reaction inference model for each of the plurality of users, and
the state learning unit does not correct the state inference model that is common.

14. The reaction generation apparatus according to claim 8, further comprising an awkwardness learning unit that generates an awkward state inference model for inferring the awkward state of the user based on the subject input information and a reaction of the subject through machine learning of a relationship of the subject input information and a reaction of the subject with the awkward state of the user.

15. The reaction generation apparatus according to claim 9, wherein
the information acquisition unit further acquires a first reaction of the subject inferred by the reaction inference model based on one piece of the subject input information, and a second reaction of the subject when one piece of the subject input information is input to the subject, and
the reaction learning unit corrects the reaction inference model based on the first reaction and the second reaction.

16. The reaction generation apparatus according to claim 2, further comprising a state learning unit that generates a state inference model for inferring a state of the subject based on the subject input information through machine learning of a relationship between the subject input information and the subject state information.

17. The reaction generation apparatus according to claim 16, wherein
the reaction of the subject is presented by a presentation unit that presents a virtual person model,
the information acquisition unit further acquires user brain wave information of a user who came into contact with the reaction presented by the virtual person model,
the state generation unit generates user state information indicating a state of the user based on the user brain wave information,
the information acquisition unit acquires, from the user, a feedback for the reaction presented by the virtual person model, when the user state information is a predetermined awkward state, and
the state learning unit corrects the state inference model based on the feedback.

18. A virtual person presentation system comprising the reaction generation apparatus according to claim 8 and a model generation apparatus that generates the virtual person model.

19. A reaction generation method comprising:
acquiring, by an information acquisition unit, subject input information input to a subject and subject brain wave information of the subject when the subject input information is input;
generating, by a state generation unit, subject state information representing a state of the subject, based on the subject brain wave information; and
generating, by a reaction generation unit, reaction information representing a reaction of the subject, based on the subject input information and the state of the subject.

20. A non-transitory computer readable medium having recorded thereon a reaction generation program that, when executed by a computer, causes the computer to perform:
an information acquisition step to acquire subject input information input to a subject and subject brain wave information of the subject when the subject input information is input;
a state generation step to generate subject state information representing a state of the subject, based on the subject brain wave information; and
a reaction generation step to generate reaction information representing a reaction of the subject, based on the subject input information and the state of the subject.
